(19) United States
(12) Patent Application Publication
(10) Pub. No.: US 2026/0071231 A1
(43) Pub. Date: Mar. 12, 2026

(54) Viral vector packaging cells with tunable virus genes to optimize vector production and quality

(71) Applicant: CHO Plus, Inc., South San Francisco, CA (US)

(72) Inventors: Kathy Ngo, San Francisco, CA (US); Larry Forman, San Mateo, CA (US)

(73) Assignee: CHO Plus, Inc., South San Francisco, CA (US)

(21) Appl. No.: 18/806,560

(22) Filed: Aug. 15, 2024

Related U.S. Application Data
(60) Provisional application No. 63/662,870, filed on Jun. 21, 2024.

(51) Int. Cl. C12N 15/86

(52) U.S. Cl. CPC C12N 15/86; C12N 2750/14143; C12N 2750/14152

(57) ABSTRACT

This disclosure provides a system for improving productivity and quality of adeno-associated virus (AAV) vectors and virus-like particles produced from packaging cell lines. Different groups of genetic elements required to produce AAV vectors (encoding regions for Rep proteins, Cap proteins, and helper function) are separated into different modules, which are each placed under control of different inducible regulatory elements. Optionally, the cell line is progeny of fused cells, and is thereby optimized for higher levels of virus production. To manufacture the intended AAV vector or particle, packaging cells containing the suite of inducible modules are contacted with (1) a payload vector containing a marker protein or therapeutic cargo, and (2) the inducer molecules for each of the regulatory elements. The viral titer, ratio of full capsids, and functional titer can be optimized by tuning the amount and ratio of each of the inducer molecules used.

### RELATED APPLICATION

This application claims the priority benefit of U.S. provisional patent application 63/662,870, filed Jun. 21, 2024. The aforesaid priority application is hereby incorporated herein by reference in its entirety for all purposes.

### TECHNICAL FIELD

The technology of this disclosure relates generally to the fields of virology, gene therapy, and production of medicaments that contain viral components. It provides a technology for the modification, selection, and genetic alteration of host cells for high levels of production of viral vectors and particles with improved biological and pharmacological characteristics.

### BACKGROUND

The past decade has seen viral vector based therapies become a bona fide option in clinical medicine. A dozen therapies using viral vectors have been approved by the FDA, spanning three different types of viral vectors: adeno-associated virus (AAV), lentivirus, and herpes simplex virus. Adenovirus vectors have been approved as immunogenic compositions for treatment of infections diseases such as COVID 19. With about 25 viral vector therapeutics currently in late-stage development and another 120 in Phase II trials, the number of viral vectors approved for commercial production will increase rapidly (E. Capra et al., Mckinsey and Company, 2022).

The first gene therapy vectors were typically developed for treatment of rare diseases. The emerging interest in treating more common conditions requires higher yields and a lower cost of goods. Over the last few years, large contract development and manufacturing organizations (CDMOs) have invested billions of dollars in production facilities for viral vectors. This burgeoning interest is promising, but the rapid influx of money and development of new technology have not solved the bottlenecks and challenges of viral vector manufacturing.

Currently, lack of standardization and low yields are part of the challenge. Physical characteristics and functional requirements vary considerably between different vectors. A high degree of process optimization is still needed for each product. Low recovery from chromatography steps means that yields are typically below 50 percent (M. May, Biotech. Eng. News, Aug. 2, 2021). By way of comparison, the manufacture of therapeutic antibodies like Humira R and Rituxan R and biosimilars is done using standardized platforms, and typically achieves yields higher than 90 percent.

The owners of the technology described in this disclosure previously developed a system for increasing production of monoclonal antibodies in producer cell lines by over four-fold (U.S. Pat. Nos. 10,329,594 and 11,649,449), and for increasing production of viral vectors by over six-fold (pre-grant publications US 2023/0399625 A1 and US 2024/0043810 A1).

This disclosure provides a further advancement in the technology for configuring vector packaging cells for high levels of production of viral vectors and particles with improved biological and pharmacological characteristics.

### SUMMARY OF THE INVENTION

This disclosure provides a system for improving productivity and quality of adeno-associated virus (AAV) vectors and virus-like particles produced from packaging cell lines. Different genetic elements required to produce AAV (encoding regions for Rep proteins, Cap proteins, and helper function) are separated into different modules, which are each placed under control of a different inducible regulatory element. Optionally, the cell line is progeny of fused cells, and is thereby optimized for higher levels of virus production. To manufacture the intended AAV vector or particle, packaging cells containing the suite of inducible modules are contacted with (1) a payload vector containing a marker protein or therapeutic cargo, and (2) the inducer molecules for each of the regulatory elements. The viral titer, ratio of full capsids, and functional titer can be optimized by tuning the amount and ratio of each of the inducer molecules used.

For purposes of this disclosure, the terms “packaging cell line” and “producer cell line” interchangeably refer to cell lines that are transiently or permanently genetically altered to produce AAV viral vectors and/or particles. The packaging or producer cell line may also be transiently or permanently genetically altered to include a payload. A “viral vector” comprises a gene that encodes a product of interest, contained in an AAV capsid. A “viral particle” comprises a protein or other gene product, contained in an AAV capsid. Depending on context, the technology of this disclosure may be used to produce either vectors or particles or both, mutatis mutandis.

Each of the “modules” is a transgene (or portion thereof) that includes at least one encoding region under transcriptional control of a regulatory element that is “tunable”. This means that the level of expression of encoding regions in the module can be increased or otherwise adjusted by the presence in the culture medium of a particular small molecule (<3,000 Da, typically <1,000 kDa, or 200 to 800 kDa), or by other condition of the culture, such as light, magnetism, or temperature. The inducing compound or condition binds to or otherwise influences an inducible regulatory element, thereby increasing or decreasing the expression of genes controlled by the respective inducible regulatory element. Each module may contain more than one encoding or regulatory region. Genes within each module may interact to invert or otherwise control gene expression or function in other modules.

Tunability of each module may or may not be reversible by reducing the concentration of the small molecule drug or reverting to previous culture conditions. For example, an inducible promoter that response to the concentration of a small molecule drug can be retuned as needed. Other inducible regulatory elements which (for example) cause gene inversion or removal of a stop codon may not be reversible after the gene has been altered. The system may be returned by taking a fresh batch of packaging cells and culturing them with the new or adjusted concentration of inducer compounds from the outset.

Different genes required for production of an AAV vector or particle include (1) AAV replication (Rep) genes: (2) AAV capsid (Cap) genes: (3) AAV assembly (aav) genes: (4) helper genes required for AAV production, and (5) a gene encoding the intended payload. Depending on context, the helper function may be supplied, for example, by one or more genes from adenovirus (Ad), specifically E1 and/or E4: by one or more genes from herpes simplex virus (HSV), specifically ICP27, ICP5, and UL5: or one or more genes from bovine papilloma virus (BVP), specifically E2A and E2B.

In the tunable technology of this disclosure, a plurality of such categories or individual genes thereof are each placed in a different module under control of a different inducible element such that the expression of such genes may be individually controlled and optimized. The plurality may be 2, 3, 4, or more than 4. Each of the modules may be but is not necessarily on a different chromosome or transgene.

Typically, the suite of transgenes is stably integrated into the genome of the packaging cell line, for example, using an integrating vector based on lentivirus, or a gene editing tool such as CRISPR. Also contemplated is a system in which a suite of three modules is transiently transfected into the cell, for example, using a DNA or RNA vector. In this case, the packaging cell line may be optimized for virus production beforehand by other means. Typically the modularized components may not include the payload vector, which can be added subsequently.

The technology of this disclosure is illustrated by a packaging cell line comprising three separately inducible modules on three separate transgenes integrated into the genome of the cells. The first module comprises at least one AAV cap gene under control of a first inducible regulatory element; the second module comprises one or a plurality of AAV helper genes under control of a second inducible regulatory element: the third module comprises at least one AAV rep gene under control of a third inducible regulatory element. Separate and not included in the packaging cell in this example is payload module that contains an expressible gene encoding a vector payload for the AAV vector or particle.

Another aspect of the technology provided in this disclosure is to provide the packaging cell with an apoptosis inhibitor such as Bcl-2, to counter pro-apoptotic effects on human host cells due to expression of one or more AAV components, such as Rep52. Rep78 or other Rep variant.

The tunable vector systems of this disclosure can be implemented together with other technologies that increase vector production and/or the proportion of filled vector capsids. For example, the packaging cells may be progeny of hybrid cells, made by fusion of two or more parental cells. Alternatively or in addition, the progeny may have been selected for high mitochondria content, or a high or low level of reactive oxygen species per cell.

The AAV vectors and particles of this disclosure usually carry a payload of nucleic acid or protein that constitutes or encodes a protein or nucleic acid of interest. To help develop and characterize the packaging cells, the vector payload can be a marker protein such as enhanced green fluorescent protein (eGFP). This can be replaced subsequently with a vector payload that is an immunogenic peptide or a nucleic acid encoding said peptide for eliciting a specific immune response in a subject in need thereof; or a nucleic acid encoding a gene product that is deficient in a subject in need of gene therapy therefor.

Once the packaging cells are built comprising a plurality of modules under separate inducible control, they can be used to optimize culture conditions to produce more vector capsids per cell and/or to increase the proportion of full capsids that include the desired payload. The tunable packaging cell line is contacted, transfected, or genetically altered with a payload vector comprising a gene that encodes a vector payload between two AAV inverted terminal repeat sequences (ITRs). The transfected cells or their progeny are cultured in the presence of different amounts and/or ratios of inducer compounds for each of said three inducible regulatory elements, thereby producing said AAV vector.

The amounts and/or the ratios of the inducer compounds in the culture medium can then be tuned or adjusted to obtain a desired titer of said AAV vector and/or proportion of viral capsids bearing said payload. In cases where the inducible regulatory element controls expression of its respective module be gene inversion or other alteration, then the consequence of the amount of inducing compound initially selected may not be reversible. Instead of tuning the packaging cells by readjusting the level of the various inducer compounds in the original culture, a fresh lot of the packaging cells is transfected with the payload vector and cultured by the new readjusted level of each of the inducer compounds at the outset.

Once optimal conditions are identified, then an industrial scale of the AAV vector or particle can be manufactured by culturing the tunable packaging cell line in a medium that contains the predetermined amount of each inducer compound for each of the multiple inducible regulatory elements; and harvesting the AAV vector from the medium. Productivity of at least 10¹¹, 10¹², and 10¹³ or more viral particles (Vp)/mL, and from 10¹⁰ or 10¹¹ up to 10¹³ or 10¹⁴ Vp/mL are contemplated. Productivity of at least 5,000, 10,000, and 20,000 VP/cell are contemplated. Ratio of full to empty capsids of at least 1, 2, and 3 are contemplated. Percent of capsids that contain payload of at least 30% 40%, 50%, and 60% are contemplated.

The selection of particular inducible regulatory elements and payloads and the construction and use of the tunable modules are detailed in the sections that follow. These and other aspects, embodiments, features, and characteristics of the invention are described below, and in the accompanying drawings and the appended claims.

### BRIEF DESCRIPTION OF THE DRAWINGS

FIG. 1 is a set of three gene maps constituting an example of how the technology provided in this disclosure can be used to construct a tunable packaging cell line for an adeno-associated (AAV) viral vector or virus-like particle. Three gene constructs or “modules” are present in the cells, interacting to form AAV vectors or particles in a tunable fashion.

FIG. 2A is a gene map of a vector comprising a payload for inclusion in the packaged AAV vector, positioned between two inverted terminal repeats (ITRs). FIG. 2B is a gene map of the product resulting from encapsulating of the payload from FIG. 2A into the packaging cell line containing Modules A. B, and C. The payload may be a marker protein or a pharmaceutical agent.

FIG. 3 is a flow diagram showing how the genetic elements mapped in FIG. 1 interact to form AAV vectors or particles in a tunable fashion. In Module B, the CreERT protein is inducible by the small molecule 4-OHT (4-hydroxytamoxifen). This causes expression of Flp5 which inverts the expression cassettes on Modules A and B. Inversion of the expression cassette on Module B results in expression of the adenovirus helper genes E4orf6 and DBP. In Module A, the EcR protein is inducible by the small molecule tebutemazide, which activates the expression cassette once inverted by Flp5. This causes expression of the AAV genes Rep52 and Cap, and also causes expression of Flp. In Module C, the Flp causes removal of the stop codon, thereby causing expression of Bcl-2 to inhibit apoptosis. Module (also comprises the AAV Rep gene under control of a TRE3G promoter, which is inducible by doxycycline.

In this example, Module A comprises a gene resistant to the antibiotic blasticidin (Bsd), Module B comprises a gene that is resistant to the antibiotic puromycin (Puro), and Module (comprises a gene that is resistant to the antibiotic hygromycin (Hygro). Culturing transfected cells in the presence of all three antibiotics will select for cells that have integrated all three modules.

FIG. 4A is a workflow for obtaining hybrid packaging cell lines that have both high virus production capacity and tunable virus components. Cells are fused together to produce an engineered cell population. The population is then cloned or separated into separate aliquots, optionally taking into account desirable cell phenotypes (such as intracellular organelle content). The separate clones or aliquots are stably transfected to express viral elements (Module A. B and (′) \and high producer cells are identified. The chosen cells are expanded to establish a bank of packaging cell lines.

FIG. 4B is a workflow for equipping a packaging cell line obtained according to FIG. 4A for production of a target AAV vector. The packaging cells are transfected with a payload vector and tuned using small molecule inducers for each of the modules. The packaging cells can be transfected to express a viral vector or particle encapsulating a therapeutic payload for gene therapy or immunization. The stable producer cell line can then be used for gene therapy or immunization without any additional transfections.

FIGS. 5A, 5B, and 5C demonstrate higher productivity of capsids of several serotypes of AAV vectors from fused HEK 293 cells. Engineered clone “44” showed 12× increase in AAV1 titer compared with parental cells. Engineered clone “2” showed 14× increase in AAV2 titer compared to parental cells. Engineered clone “121” showed 5× increase in AAV5 titer compared to parental cells. Engineered clone “17” showed 6× increase in AAV9 titer compared to parental cells.

FIGS. 6A, 6B, and 6C show how modules may be tuned to optimize virus production and phenotype. Packaging cell lines were made from HEK 293 cells (“parent”), or from progeny of HEK 293 cells fused together (“hybrids”). The packaging cell lines were genetically altered to incorporate the three tunable modules shown FIG. 1.

FIG. 6A shows the conditions of a trial expression culture. The medium contained a relatively low level of tebufenozide (inducer A), a moderate level of 4 hydroxytamoxifen (4 OHT, inducer B), and a relatively high level of doxycycline (inducer C). The tuned cells were transfected with the AAV payload (GFP), grown in the adapted culture medium, and virus was harvested. FIG. 6B shows the capsid titer obtained. Using separate packaging cells made with cell hybrids, serotypes AAV1, AAV2, and AAV5 were produced at a level that was as much as 4-fold higher or more, compared with packing cells made from the parental HEK 293 line. FIG. 6C shows the percentage of capsules that were full (contained the intended payload).

FIGS. 7A, 7B, and 7C show another trial in which the concentrations of inducers A and B were higher than in FIGS. 6A, 6B, and 6C, whereas the concentration of inducer C was lower. There was a lower capsid titer (FIG. 7B), but a higher percentage of full capsids (FIG. 7C).

### DETAILED DESCRIPTION

This disclosure provides improved virus packaging cell lines for producing viral vectors and virus-like particles for delivering nucleic acid and protein respectively into a target cell.

### 1. OVERVIEW AND SELECTED EXAMPLE OF A TUNABLE VECTOR PACKAGING CELL

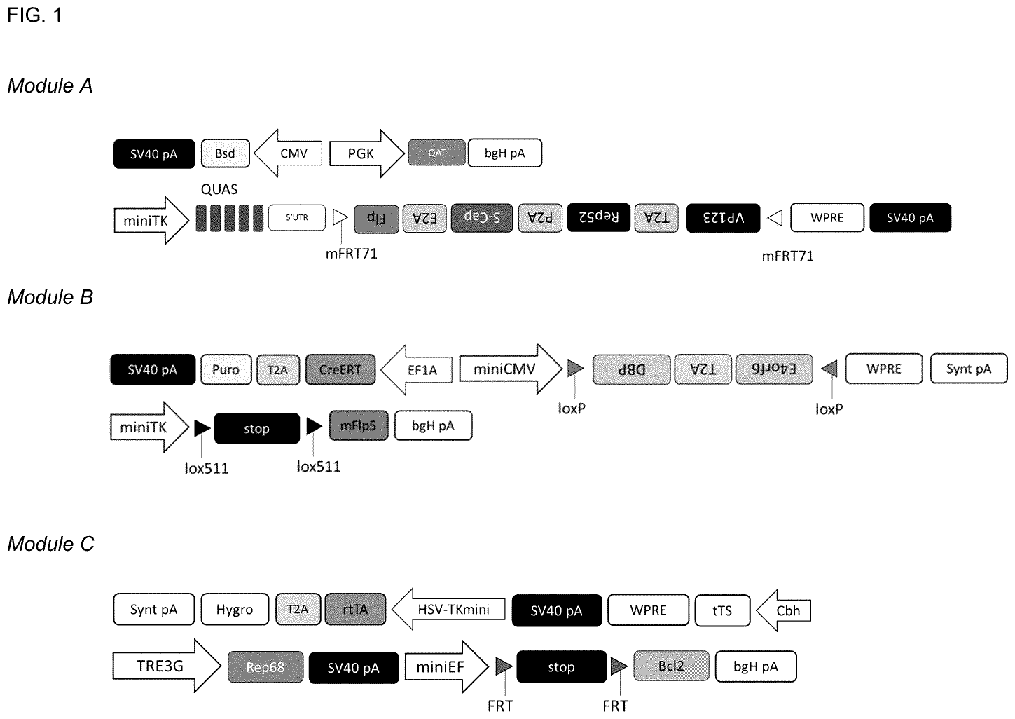

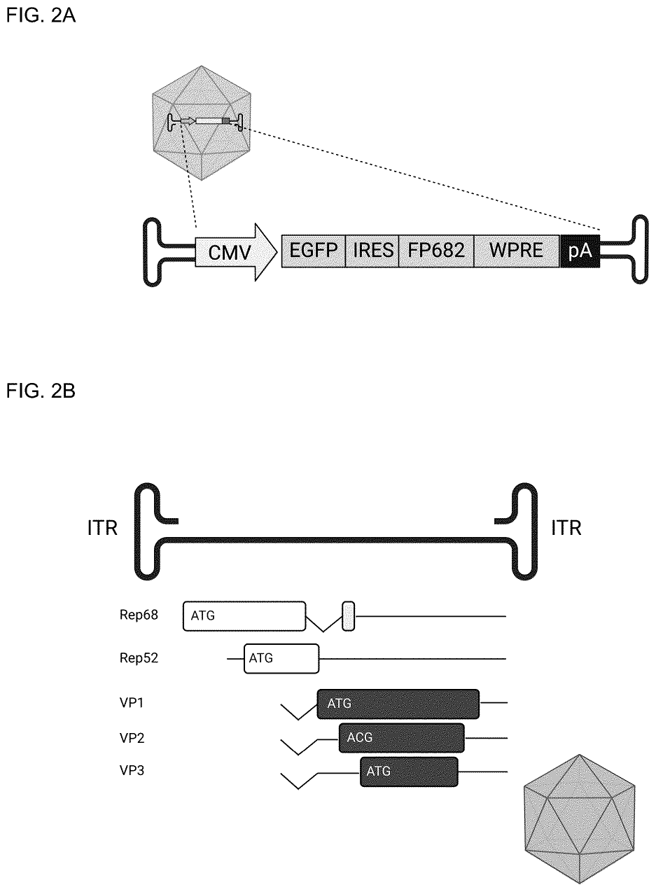

FIG. 1 is a gene map of a non-limiting implementation of this technology. Three modules are introduced into the virus producer or packaging cell line, which interact for separate and tunable expression of AAV Rep and Cap genes, and adenovirus helper function. A map of a suitable payload vector for delivery into the target cell by the vector is shown in FIG. 2A. A map of the vector manufactured by the packaging cell including the payload is shown in FIG. 2B.

This system can be used to produce viral titer of the level of 10¹² Vp/mL Features of this system are as follows:
Replication, cap, and helper components are in 3 independent modules of the host cell:
Each module expression is dependent on a single small molecule inducer, which can be used to tune expression:
Helper functions are divided into Module A and Module B to allow for finer control of VP proteins:
Replication proteins are divided into Module A and Module C for tuning either for higher titer and/or higher percent full capsid:
In other systems, overactivation of Rep52 can result in cell death. The effect is minimized in this system by the presence of the apoptosis-inhibiting gene Bcl-2.

FIGS. 2A and 2B are gene maps showing a prototype payload vector suitable for encapsulating in an AAV vector, and the AAV vector product made therefrom. The payload vector is a bicistronic construct for co-expressing both enhanced green fluorescent protein (eGFP) and infrared fluorescent protein 682 (iFP682). The two fluorescent reporters mimic therapeutic payload size used for gene therapy (˜3-4 kb). A strong ubiquitous promoter (CMV) was used to express EGFP and FP682. WPRE was used to enhance transgene expression, while poly A from SV40 (pA) ensures proper transcription termination by adding poly A tail to transcribed mRNA. Shown in FIG. 2B is a genome map of AAV capsid (bottom panel) encapsulated with payload (top panel) into packaging cell line containing Modules A, B, and C.

The AAV capsid produced thereby contains recombinant Rep and Cap (VP1, VP2, and VP3) open reading frames and inverted terminal repeats (ITRs). To ensure correct capsid protein stoichiometry for VP1:VP2:VP3 of 1:1:10, VP1-3 capsid protein production requires both alternative mRNA splicing as well as alternative translational start codons. VP1 is translated from ATG start codon; while VP2 uses a weaker ACG start codon and readthrough translation to the next available ATG codon for the production of the most abundant capsid protein, VP3. A schematic of encapsulated virus particle containing payload is shown in the lower bottom corner of FIG. 2B.

Abbreviations used in the gene maps are as follows:

### Module A:

SV40 pA=simian virus 40 polyadenylation signal
Bsd=blasticidin S deaminase: selectable marker gene conferring resistance to the antibiotic blastocidin
CMV=cytomegalovirus promoter: drives high-level gene expression
PGK=phosphoglycerate kinase promoter: strong and constitutive gene expression
CFDBD=quinic acid fucose dimeric biosensor domain: detects specific molecules or ligands in cellular environments
AD=activation domain: interacts with transcriptional machinery to enhance gene expression
EcR=enhancer core region: promotes the assembly of transcriptional machinery
bgH pA=bovine growth hormone polyadenylation signal
miniTK promoter=minimal thymidine kinase promoter: drives transgene expression
QUAS=QF-responsive upstream activating sequence for controlling gene expression
5′UTR=5′ untranslated region of QUAS gene
mFRT71=modified FRT71: modified version of the FRT (flippase recognition target) site, used for site-specific recombination
Flp=flippase: enzyme for catalyzing site-specific recombination between FRT sites
E2A=sequence from adenovirus 2 that enhances transcription when bound by transcription factors
S-Cap=AAV gene (capsid)
T2A=Thosea asigna virus 2A: sequence from Thosea asigna virus that mediates ribosome skipping, for expression of multiple proteins from a single mRNA transcript
Rep52=AAV gene (viral DNA replication and regulation of gene expression)
T2A=Thosea asigna virus 2A: sequence from Thosea asigna virus that mediates ribosome skipping, for expression of multiple proteins from a single mRNA transcript
VP1/VP2/VP3 (VP123)=capsid protein complex that self-assembles to form icosahedral capsid: promotes viral entry into host cells by binding to host cell heparan sulfate and is a key target for the host immune response
WPRE=woodchuck hepatitis virus posttranscriptional regulatory element: enhances stability and translation efficiency of mRNA transcripts in mammalian cells
SV40 pA=simian virus 40 polyadenylation signal

### Module B:

SV40 pA=simian virus polyadenylation signal
Puro=Puromycin resistance gene, a selectable marker gene conferring resistance to the antibiotic puromycin
T2A=Thosea asigna virus 2A: sequence from Thosea asigna virus that mediates ribosome skipping, for expression of multiple proteins from a single mRNA transcript
CreERT=a fusion protein combining the Cre recombinase enzyme with the ligand-binding domain of the estrogen receptor. It allows for inducible and temporally controlled activation of Cre-mediated recombination in response to tamoxifen
EF1A=Eukaryotic elongation factor 1 alpha promoter, drives high-level and constitutive gene expression
miniCMV=minimal cytomegalovirus promoter: drives transgene expression
miniTK=minimal thymidine kinase promoter: drives transgene expression
loxP=LoxP site recognized by Cre recombinase
lox511=mutated Lox sites recognized by Cre recombinase
DBP=DNA-binding protein
T2A=Thosea asigna virus 2A: sequence from Thosea asigna virus that mediates ribosome skipping, for expression of multiple proteins from a single mRNA transcript
E4orf6=early region 4 open reading frame 6, an adenovirus gene that encodes a multifunctional protein that modulates host cell processes to facilitate viral replication
WPRE=woodchuck hepatitis virus posttranscriptional regulatory element: enhances stability and translation efficiency of mRNA transcripts in mammalian cells
Synt PA=synthetic polyadenylation signal
miniTK promoter=minimal thymidine kinase promoter: drives transgene expression
Stop=stop codon
mFlp5=modified version of the Flippase (Flp) enzyme for catalyzing site-specific recombination between FRT sites
bgH pA=bovine growth hormone polyadenylation signal

### Module C:

Synt PA=synthetic polyadenylation signal
Hygro=hygromycin resistance gene, a selectable marker gene conferring resistance to the antibiotic hygromycin
T2A=Thosea asigna virus 2A: sequence from Thosea asigna virus that mediates ribosome skipping, for expression of multiple proteins from a single mRNA transcript
rtTA=reverse tetracycline-controlled transactivator: transcriptional activator protein that is regulated by tetracycline for inducible gene expression
HSV-TKmini=truncated version of the Herpes simplex virus thymidine kinase promoter for driving transgene expression
SV40 pA=simian virus polyadenylation signal
WPRE=woodchuck hepatitis virus posttranscriptional regulatory element: enhances stability and translation efficiency of mRNA transcripts in mammalian cells
tTS=tetracycline-controlled transcriptional silencer: regulated by tetracycline for inducible gene silencing
Cbh=chicken beta actin hybrid promoter for constitutive gene expression
TRE3G=tetracycline-responsive element, third generation (3G), responds to tetracycline for inducible gene expression in mammalian cells
Rep68=AAV Replication protein 68: A protein encoded by the Rep gene of AAV, involved in regulation of viral gene expression
SV40 pA=simian virus polyadenylation signal
miniEF=minimal elongation factor 1 alpha promoter: for driving gene expression
FRT=Flippase recognition target for Flp-FRT-mediated gene excision or integration
Stop=stop codon
Bcl-2=B-cell lymphoma/leukemia-2 gene: inhibits apoptosis (programmed cell death)
bgH pA=bovine growth hormone polyadenylation signal

The function of these components in the expression system is shown in Table I.

TABLE 1
Function of viral elements in Modules A, B, and C

| | Full Name | Function |
|---|---|---|
| SV40 pA | Poly A tail for simian | Stabilize mRNA and proper exportation from nucleus to |
| | virus 40 | cytoplasm for protein translation |
| bGH pA | Bovine growth hormone | Stabilize mRNA and proper exportation from nucleus to |
| | polyadenylation signal | cytoplasm for protein translation |
| Synt pA | Synthetic poly A | Stabilize mRNA and proper exportation from nucleus to |
| | | cytoplasm for protein translation |
| WPRE | Woodchuck Hepatitis | Enhance transgene expression |
| | Virus Posttranscriptional | |
| | Regulatory Element | |
| Cbh | CMV enhancer and | Ubiquitous, strong promoter driving transgene |
| | chicken β-actin hybrid | expression |
| | promoter | |
| TRE3G | Tetracycline regulatory | Inducible promoter that is activated upon binding of rtTA |
| | element, third generation | and addition of doxycycline activating Rep68 |
| CMV | Cytomegalovirus | Ubiquitous, strong promoter driving transgene |
| | promoter | expression |
| PGK | 3-phosphoglycerate | Ubiquitous, weak promoter driving transgene expression |
| | kinase promoter | |
| EF1A | Human elongation factor- | Ubiquitous, strong promoter driving transgene |
| | 1 alpha | expression |
| miniCMV | Minimal cytomegalovirus | Minimal weak promoter driving transgene expression |
| | promoter | |
| miniTK | Minimal thymidine kinase | Minimal weak promoter driving transgene expression |
| | promoter | |
| HSV-TKmini | Minimal herpes simplex | Minimal weak promoter driving transgene expression |
| | virus (HSV) thymidine | |
| | kinase promoter | |
| miniEF | Minimal human | Minimal weak promoter driving transgene expression |
| | elongation factor-1 alpha | |
| | promoter | |
| Bsd | Blasticidin | Antibiotic selection marker for Module A |
| Hygro | Hygromycin | Antibiotic selection marker for Module B |
| Puro | Puromycin | Antibiotic selection marker for Module C |
| QAT | QFDBD-EcRAD fusion | Fusion protein containing: 1) DNA binding domain of QF |
| | protein | or qa-1F transcription factor; and 2) VP16 activation |
| | | domain fused to modified Ecdysone receptor that is |
| | | activated upon addition of tebufenozide ligand (Teb). |
| | | Upon addition of Teb, QAT is activated and binds to |
| | | QUAS to activate any transgene downstream of QUAS. |
| QUAS | QF upstream activation | Upon binding of QAT to QUAS, any downstream gene is |
| | sequence | activated |
| 5′UTR | 5′ untranslated region | Enhance translation efficiency of messenger RNA |
| Flp | Flippase | Recombinase that catalyzes homologous recombination |
| | | at two Flippase recognition targets (FRT) activating Bcl-2 |
| | | gene to inhibit cell death |
| mFlp5 | mutant Flippase 5 | Mutant Flippase recombinase that catalyzes homologous |
| | | recombination at Flippase recognition target sites, |
| | | mFRT71, activating E4orf6 and DBP |
| FRT | Flippase recognition | Homologous recombination sites FRT catalyzed by Flp |
| | targets | |
| mFRT71 | Mutant Flippase | Homologous recombination sites mFRT71 catalyzed by |
| | recognition targets 71 | mFlp5 |
| CreERT | Cre recombinase (Cre) | Fused protein that is activated upon addition of |
| | fused to a mutant | 4-hydroxytamoxifen (4-OHT) |
| | estrogen ligand-binding | |
| | domain (ERT2) | |
| loxP | Locus of X-over P1 | 34 base pair DNA sequence recognized by Cre |
| | | recombinase to induce homologous recombination |
| lox511 | Locus of X-over 511 | Mutant loxP site recognized by Cre recombinase that can |
| | | result homologous recombination with another lox511 |
| | | site |
| rtTA | Reversible tetracycline | Transcription activator that requires the addition of |
| | transcriptional activator | doxycycline. rtTA binds to TRE site to activation |
| | | transgene downstream of TRE |
| tTS | Tetracycline controlled | Reduces the leakage of gene activation in the absence |
| | transcriptional silencer | of doxycycline |
| Stop | Stop cassette | Terminate gene transcription |
| E2A | 2A peptide from equine | Allows for polycistronic gene expression: transcription of |
| | rhinitis A virus | multiple genes from a single open reading frame |
| P2A | 2A peptide from porcine | Allows for polycistronic gene expression: transcription of |
| | teschovirus-1 | multiple genes from a single open reading frame |
| T2A | 2A peptide from thosea | Allows for polycistronic gene expression: transcription of |
| | asigna virus | multiple genes from a single open reading frame |
| S-Cap | Serotype-specific capsid | Capsid genes essential for forming proper AAV capsid |
| | genes | |
| Rep52 | Rep52 | Required for proper formation of AAV capsid |
| Rep68 | Rep68 | Required for proper formation of AAV capsid |
| VP123 | VP1, VP2, and VP3 | Structural protein for AAV |
| E4orf6 | E4orf6 | Required for transportation of viral RNA |
| DBP | D-Box Binding PAR BZIP | Enhance posttranslational expression of AAV |
| | Transcription Factor | |
| Bcl-2 | B-cell lymphoma 2 | Cell death inhibitor |

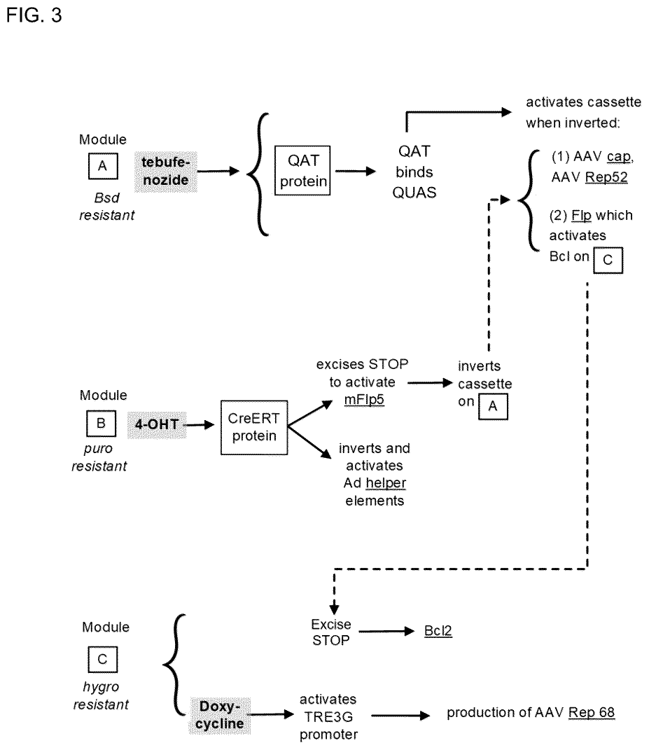

FIG. 3 is a flow chart showing functional interaction and flow of the vector system mapped in FIG. 1. Starting with Module B, the CreERT protein is a gene inverting enzyme, comprising Cre recombinase and estrogen receptor ligand binding domain. It responds to the small molecule inducer 4-hydroxytamoxifen (4-OHT) by activating recombinase activity. This acts on Module B to invert and activate the expression cassette bounded by two loxP sites to put the cassette in an expressible configuration. CreERT also excises the stop codon between the two lox511 sites on Module B to activate expression of mFlp5.

Module A is under control of QAT fusion protein. The QAT fusion protein contains an activation domain of VP16 fused to modified but truncated Ecdysone receptor (EcR), which responds to the small molecule inducer tebufenozide. Activation of QAT allows binding QFDBD domain to QUAS, causing gene expression from the expression cassette that was inverted by Module B—which, in turn, causes expression of the Cap and Rep52 proteins for assembly into the viral vector. QAT also activates the other recombinase Flp, which removes the stop codon on Module C.

Module C has two components. One is under control of the small molecule inducer doxycycline. It activates the inducible promoter TRE3G, which causes expression of Rep68, the other component needed for viral production. Also residing on Module C is the human apoptosis inhibitor gene Bcl-2. This is included in the system to counter the overexpression of Rep52, which can cause death, resulting in reduced viral production in the packaging cell.

Put another way, Module A is a packaging module that contains some components of Rep and Cap. S-Cap is the serotype specific capsid used to package each AAV serotype, under the control of tebufenozide. Upon expression of mFlp5 (a variant of Flp recombinase that only induces homologous recombination at mFRT71 sites) from Module B and 4-OHT. This results in expression of VP1/VP2/VP3, Rep52, and serotype-specific cap protein (S-Cap). Tebufenozide binds QAT, activating QF transcriptional activator (QFDBD), which then binds to QUAS enhancer sites. This in turn promotes inversion of cassette containing VP1/VP2/VP3, Rep52, S-Cap, and Flp.

Module B contains components E4orf6 and DBP to allow for sufficient helper functions. Helper function is under the control of 4-OHT. Expression of mFlp5 is normally OFF and activated upon induction of 4-OHT, resulting in Cre expression, excising the stop codon upstream of mFlp5, resulting in its expression.

Module C contains the gene encoding Rep68, which confers a viral replication function under the control of doxycycline. Module C also contains the human Bcl-2 gene under control of the Flp enzyme on Module A. In some previous systems, Rep52 overactivation resulted in apoptosis (cell death) of the packaging cell. In this example, Rep52 is linked to Flp, which induces homologous recombination at FRT sites in Module C, which leads to activation of Bcl-2, inhibiting apoptosis.

### 2. PROCEDURE FOR BUILDING PACKAGING CELL LINES

Selecting parental cell line for producing packaging cells according to this technology is influenced by viral type, and whether the cell line has elements that may be missing in a replication deficient virus. Suitable cell lines for adeno-associated virus (AAV): HEK 293, HeLa, A549, BHK-21, Vero: Adenovirus: HEK 293, HeLa, A549, COS-7, Vero; Herpes simplex virus (HSV): Vero, HeLa, BHK-21, CHO-K1, NIH/3T3.

Potential parental cells are as follows:
Vero cells, derived from African green monkey kidney tissue, ATCC #CCL-81:
HEK 293 cells, originating from human embryonic kidney tissue, ATCC #CRL-1573:
HeLa cells, derived from cervical cancer cells, ATCC #CCL-2:
A549 cells, derived from human lung carcinoma tissue, ATCC #CCL-185:
BHK-21 cells, derived from baby hamster kidney tissue, ATCC #CCL-10:
COS-7 cells, originating from African green monkey kidney cells transformed with SV40, ATCC #CRL-1651;
NIH/3T3 cells, derived from mouse embryonic fibroblasts, ATCC #CRL-1658.

By way of illustration, a packaging system using the modules shown in FIG. 1 can be produced as follows:
1. Linearize plasmid DNA containing Module A, B, and C by restriction enzyme digest and purify:
2. Grow HEK 293 cells in shake flask so that they are in log phase:
3. Triple transfect cells by electroporation with linearized DNA containing Module A, B, and C:
4. Grow cells in selection medium containing blasticidin, hygromycin, puromycin for 3 weeks in shaking culture flasks to select for cells that have integrated all three modules.
5. Prepare frozen aliquots of stable cell pool expressing Module A, Module B, Module C.

### 3. PROCEDURE TO USE PACKAGING CELL LINES AND TO CHARACTERIZE VECTORS AND PARTICLES OBTAINED THEREBY

Step 1. Transfection and small molecule induction:
1. Thaw vial of frozen HEK 293 cells expressing Module A, Module B, and Module C:
2. Grow HEK 293 cells to VCD of 3×10⁶ cells/mL in growth media without antibiotics;
3. Transfect HEK 293 cells by chemical-based methods with transfer vector containing payload flanked by AAV inverted terminal repeats:
4. Culture cells for 4 h at 37 C, 8% CO₂, and 125 rpm;
5. Add tebufenozide (4-OHT) and doxycycline to transfected cells.
6. Culture cells for additional 2 at 37 C, 8% CO₂, and 125 rpm;

Step 2. Lyse and harvest cells:
7. After 72 hours, add benzonase and lysis buffer to transfected cells. Culture cells for 2 h at 37 C, 8% CO₂, and 125 rpm:
8. Harvest lysed cells by centrifugation at 4000 rpm at 4° C. for 30 min;
9. Transfer supernatant containing AAV particles to new centrifuge tube to assay for AAV production.

Step 3: Assay for viral genome titer
10. Measure AAV genomic copy number by real-time quantitative PCR. Treat sample with DNase I to remove non-viral host genomic DNA:
11. Real-time quantitative PCR by fluorescent detection performed using DNA primers that bind to coding regions of fluorescent reporter within transfected transfer vector;
12. Assay for viral genome titer.

Step 4: Measure concentration of AAV serotype-specific capsids:
13. Bio-layer interferometry (BLI) is an optical biosensing technology that analyzes biomolecular interactions in real-time without the need for fluorescent labeling. Interference patterns of white light or phase shift caused by analyte sample binding to immobilized ligand on biosensor probe was used to quantify the amount of AAV virus in an unknown sample. (The apparatus comprises a small biosensor that binds specifically to AAV capsid protein for multiple serotypes (AAV1, AAV2, and AAV5). For each serotype (AAV1, AAV2, and AAV5), a standard curve of commercial AAV reference standing with known capsid concentration measured by other validated methods are used to back-calculate the concentration of AAV serotypes in unknown sample.)

Step 5: Measure the ratio of full to empty capsids:
14. Ratio is measured in unknown samples by biolayer interferometry:
15. Determine AAV serotype-specific capsid concentration of AAV as described above:
16. For each serotype being measured, AAV capsid at a normalized concentration are first captured and immobilized on biosensor:
17. Following immobilization, AAV particles are lysed to release packaged single stranded DNA (ssDNA) and ssDNA is captured and measured using biosensor probe that is conjugated to SSB protein wherein SSB protein binds specifically to only ssDNA:
18. Perform binding kinetics assay of unknown samples and commercial AAV reference standard, with known full-to-empty capsids ratio measured by other validated methods:
19. Back-calculate the ratio of AAV serotypes in unknown sample using commercial AAV reference standard, with known full-to-empty capsids ratio.

Step 6: Measure functional titer:
20. HEK 293 cells are infected with undiluted samples of AAV1, AAV2, and AAV5 at a range of dilutions and added to a fixed population of HEK 293 cells:
21. Functional titer is measured in transduction units (TU) per mL. Infectivity was determined by quantifying percentage of fluorescent-positive cells by flow cytometry at different multiplicity of infection (MOI).

### 4. PROCEDURE FOR TUNING THE PACKAGING CELL TO MAXIMIZE VIRAL CAPSID PRODUCTION AND PAYLOAD INCORPORATION

The user may employ the packaging cells of this invention to optimize production of AAV vectors or virus like particles, for example, in terms of viral particles per cell or per mL culture fluid, or in terms of the percent of viral capsids that contain a payload. Different modules or cassettes in the cell may be dialed up or down by adjusting the amount of the inducer element. By way of illustration, the system mapped in FIG. 1 can be dialed as shown in Table 2.

TABLE 2
Dialable components of the modules

| | Controlled by | | | |
|---|---|---|---|---|
| Module | inducer | Dial | Activates | Function |
| A | tebufenozide | Teb Dial containing | Rep52, S-Cap, VP1, | Packaging: Produces |
| | | QAT cassette | VP2, VP3, and Flp | packaging genes and Flp |
| B | 4-OHT | OHT Dial containing | E4orf6, DBP, and | Helper: Produces helper |
| | | CreERT2 | mFlp5 | proteins and mFlp5 |
| C | doxycycline | Dox Dial containing | Rep68 and Bcl-2 | Replication: inhibits apoptosis |
| | | TRE3G | | when Rep52 is too high and |
| | | | | increase viral capsid |
| | | | | production |

Module A is a packaging module that contains some components of Rep and Cap. S-Cap is the serotype specific capsid used to package each AAV serotype. It is under the control of tebufenozide. Upon expression of mFlp5 (a variant of Flp recombinase that only induces homologous recombination at mFRT71 sites) from transfer vector and tebufenozide. Expression of VP1/VP2/VP3, Rep52, and serotype-specific cap protein (abbreviated as S-Cap). Tebufenozide binds the EcRAD fusion protein (truncated ecdysone receptor fused to activation domain of VP16), activating QAT fusion protein. QAT then binds to QUAS enhancer sites, promoting the inversion of cassette containing VP1/VP2/VP3, Rep52, serotype-specific cap protein (abbreviated as S-Cap), and Flp. As Rep52 overactivation can result in cell death: Rep52 is indirectly linked to Flp, which induces homologous recombination at FRT sites in Module C, activating a cell death inhibitor, Bcl-2.

Module B contains components E4orf6 and DBP to allow for sufficient helper functions. Helper function Is under the control of 4-OHT. Expression of mFlp5 is normally OFF and activated upon induction of 4-OHT, resulting in Cre expression, excising the stop codon upstream of mFlp5, resulting in its expression.

Module C: contains components additional Rep protein, Rep68 (function only viral replication machinery), Flp recombinase protein, and hBCL2 (under the control of Flp expression in Module A). The level of Rep68 expression is under the control of doxycycline.

### Tuning Modules a, B, and C for High Viral Titer, High Percent Filled Ratio, and or High Functional Titer

Once the packaging cell has been constructed, it is used to produce AAV vectors as follows. First, the cells are transfected with a transfer vector containing a payload. The transfected cells are tuned with a measured concentration of the respective small molecule inducer:
1. Thaw vial of frozen HEK 293 cells expressing Module A, Module B, and Module C.
2. Grow HEK 293 cells to VCD of 3×10⁶ cells/mL in growth media without antibiotics.
3. Transfect HEK 293 cells by chemical-based methods with transfer vector containing payload flanked by AAV inverted terminal repeats.
4. Culture cells for 4 h at 37 C, 8% CO₂, and 125 rpm.
5. Add tebufenozide at concentration range of 1 μM-10 μM to transfected cells.
6. Add 4-OHT at concentration range of 1-10 μM to transfected cells.
7. Add doxycycline at concentration range 100-500 μg/mL to transfected cells.
   Subsequent steps include lysis and harvesting of the cells, assaying for viral genome titer, measuring AAV serotype specific capsids, measuring ratio of full to empty capsids, and measuring functional titer, as described above. Depending on the results, a fresh lot of packaging cells can be retuned and remeasured.

The drawings provide two examples in which Modules A. B, and C were variously tuned to increase capsid titer or the percentage of full capsids made by HEK 293 producer cells.

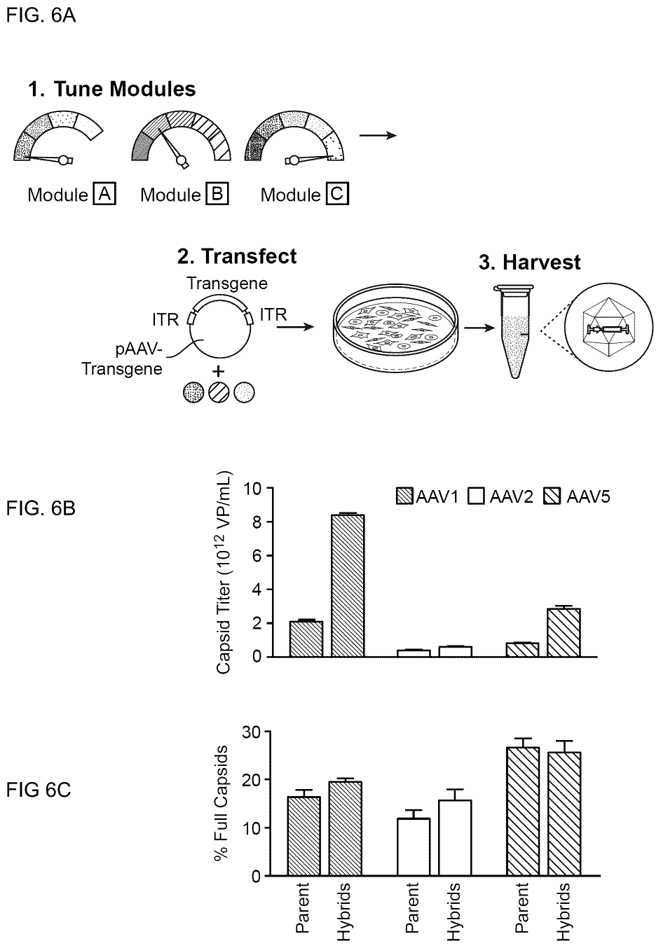

FIGS. 6A to 6C is a first example in which modules were tuned to increase virus production. In FIG. 6A, packaging cells containing the three modules were introduced with a payload vector. Module A, B, and C were turned to levels of low, low-medium, and high using the small molecule inducers Teb, O H T, and Dox, respectively. FIGS. 6B and 6C compare capsid titer and the percentage of full capsids measured in supernatants for cells adapted to package three different serotypes of AAV: specifically, AAV1, AAV2, and AAV5. Tuning the modules in this fashion increased capsid titer up to 4-fold while maintaining similar percentage of full capsids.

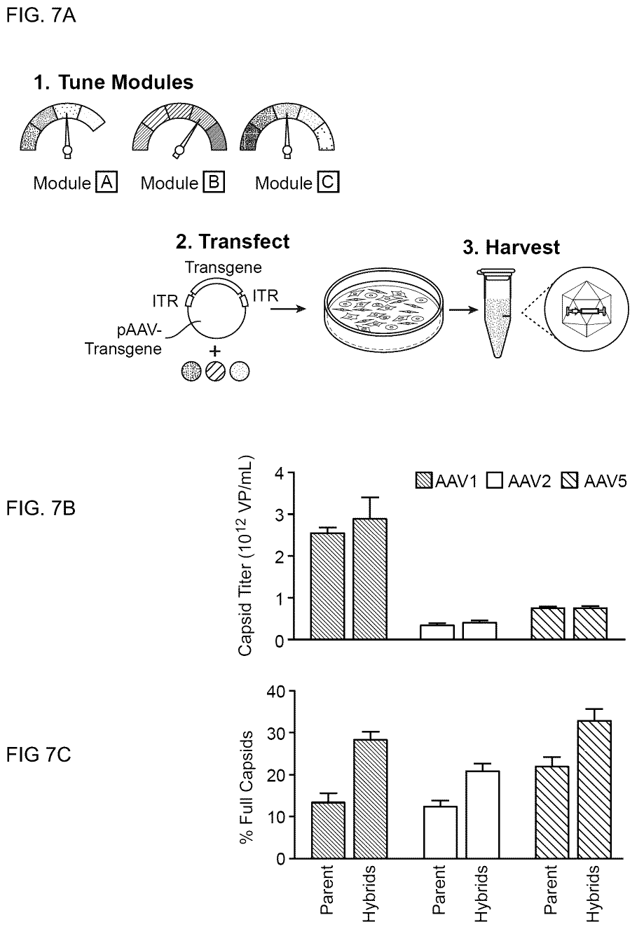

FIG. 7A to 7C show a second example in which Modules A. B, and C were tuned to produce a higher percentage of full capsids. In FIG. 7A, packaging cells containing the three modules were further transfected with a payload vector. Modules A, B, and C were turned to levels of medium, medium-high, and medium using the small molecule inducers Teb, OHT, and Dox dials, respectively. FIGS. 7B and 7C compares capsid titer and percent full capsids measured in supernatants harvested from packaging cells for the three AAV serotypes. Tuning the modules in this manner increased the percentage of full capsids produced from packaging cells made with hybridized parental cells.

### 5. INDUCIBLE REGULATORY ELEMENTS FOR USE IN A TUNABLE SYSTEM

A hallmark of the tunable packaging cells in this disclosure is the use of multiple genetic elements that responds to small molecule inducers (or other effects that can be imposed upon culturing of the cells).

The inducible element used in Module A (FIG. 1) is the hybrid bipartite QF and ecdysone receptor (ECR) system: two un-related truncated proteins fused together for controlling gene expression in a regulated manner. QFDBD serves as a transcription factor that binds to Q-response elements (QREs) but requires activation of Teb ligand which binds to truncated Ecdysone receptor (EcR). Other singular and bi-partite regulatory elements, which offer inducible gene expression, include:
Tetracycline-Inducible Systems: These systems are based on the tetracycline repressor (TetR) protein and its interaction with tetracycline or its derivatives (e.g., doxycycline). Examples include Tet-On and Tet-Off systems.
Lac Operon/Isopropyl β-D-1-thiogalactopyranoside (IPTG)-Inducible Systems: These systems utilize the Lac repressor (Lacl) protein and its interaction with the inducer IPTG to control gene expression.
Cre-LoxP Recombination System: This system involves the Cre recombinase enzyme and its ability to catalyze site-specific recombination between DNA sequences known as LoxP sites. Inducible versions of this system can be controlled by promoters that regulate Cre expression.
Flp-FRT Recombination System: Similar to the Cre-LoxP system, this system involves the Flp recombinase enzyme and its ability to catalyze recombination between FRT sites.
GeneSwitch® System: This system utilizes a fusion protein comprising a mutant progesterone receptor ligand-binding domain and a transcriptional activator domain. Gene expression is induced by the synthetic ligand, mifepristone (RU486).
Chemically-Inducible Systems: These systems use small molecules or chemical compounds as inducers to regulate gene expression. Examples include the rapamycin-inducible system (based on the FK506 binding protein (FKBP) and rapamycin) and the Shield-1-inducible system (based on the FKBP protein and Shield-1 ligand).
Heat Shock-Inducible Promoters: These promoters drive gene expression in response to elevated temperatures, typically in the range of 37-42° C.

The inducible element in Module B is CreERT. The CreERT protein is a fusion protein that combines the Cre recombinase enzyme with the ligand-binding domain of the estrogen receptor (ER). This fusion protein allows for inducible and temporally controlled activation of Cre-mediated recombination in response to the estrogen receptor agonist, typically tamoxifen or its active metabolite 4-hydroxytamoxifen (4-OHT). When tamoxifen or 4-OHT binds to the estrogen receptor domain of CreERT, it induces a conformational change in the protein, activating its recombinase activity. This activated CreERT can then catalyze site-specific recombination between loxP sites, leading to the deletion, inversion, or translocation of DNA sequences flanked by loxP sites.

The inducible element in Module C is the Tetracycline-Responsive Element 3G (TRE3G) promoter: a synthetic DNA sequence engineered to regulate gene expression in response to the presence or absence of tetracycline or its derivative, doxycycline.

More generally, inducible elements that can be used to control virus gene expression include the following:
Promoters: Promoters are DNA sequences that initiate the transcription of a gene. In viral vectors, viral promoters (e.g., CMV promoter, SV40 promoter) or tissue-specific promoters (e.g., promoters specific to liver, muscle, or neuronal cells) are often used to control where and when the transgene is expressed.
Enhancers: Enhancers are DNA sequences that can increase the transcriptional activity of a promoter. They can be viral enhancers or cellular enhancers, and they contribute to fine-tuning the expression levels of the transgene.
Polyadenylation (polyA) signal: The polyA signal is a sequence of RNA that signals the termination of mRNA synthesis and the addition of a polyadenine tail to the mRNA. It enhances mRNA stability and translation efficiency.
Transcriptional terminators: Transcriptional terminators are sequences that signal the end of transcription. They help prevent read-through transcription from adjacent sequences and ensure proper processing of mRNA.
Regulatory elements for viral replication and packaging: These elements are necessary for viral vector replication and packaging. They include sequences essential for viral genome replication, packaging signals, and sequences required for viral particle assembly.
Response elements: Response elements are sequences that respond to specific cellular or environmental signals, allowing for inducible or regulated expression of the transgene. Examples include tetracycline-responsive elements (TRE), doxycycline-inducible promoters, or heat shock-responsive elements.
Insulators: Insulators are DNA sequences that can block the effects of nearby enhancers or silencers, preventing inappropriate activation or repression of neighboring genes.

The inducible elements in the modules shown in FIG. 1 are as follows:

QAT fusion protein consisting of QF DNA-binding domain combined with truncated EcR protein fused to VP16 activation domain. QF serves a transcription factor while truncated EcR enables the ligand-receptor activation by Teb. QF is under the category of transcription factors or regulatory proteins. It should be noted the QF can be activated by quinic acid but this response to quinic acid have been abrogated in QAT fusion protein in Module A.

CreERT protein is a fusion protein that consists of two main parts: Cre recombinase and a modified estrogen receptor ligand-binding domain (ERT). The Cre recombinase recognizes specific DNA sequences known as loxP sites and induces recombination between them, leading to DNA rearrangement. The ERT domain allows the activity of Cre recombinase to be controlled by the presence or absence of tamoxifen or its derivatives. Thus, the CreERT is a type of inducible regulatory protein.

TRE3G promoter contains tetracycline operator (TetO) sequences that bind to the tetracycline-controlled transcriptional activator (tTA) or reverse tetracycline-controlled transactivator (rtTA) protein in the absence or presence of tetracycline or its derivatives. This system allows for tight control of gene expression in response to the presence or absence of the inducer molecule. Thus, TRE3G promoter is an inducible regulatory DNA element.

Other inducible or regulatory elements that can be used in the context of this technology include the following:

### Transcription Factors:

Gal4: Utilized in the Gal4-UAS system for inducible gene expression.
LexA: Another transcription factor used in inducible gene expression systems.
TetR: The tetracycline repressor protein, used in tetracycline-inducible systems.

### Recombinases:

FlpERT: Similar to CreERT, FlpERT is a fusion protein comprising Flp recombinase and a modified estrogen receptor ligand-binding domain, enabling conditional gene manipulation.
Split-Cre Systems: Divides Cre recombinase into two inactive fragments, reassembling and becoming active in the presence of specific inducers.
Optogenetic Recombinases: Light-inducible recombinases activated by specific wavelengths of light, offering precise spatiotemporal control over genetic manipulation.

### Promoters:

Tet-On/Tet-Off Systems: Utilize tetracycline-controlled transcriptional activators (tTA/rtTA) along with tetracycline-responsive promoters for inducible gene expression.
RU486-Inducible Promoters: Responsive to the synthetic steroid mifepristone (RU486) for controlled gene expression.
Gal4-UAS System: Relies on the yeast transcription factor Gal4 and its upstream activating sequence (UAS) for inducible gene expression.

### Chemically-Inducible Systems:

RU486-Inducible Systems: These systems use the synthetic steroid mifepristone (RU486) to control gene expression through engineered ligand-responsive promoters.
Ecdysone-Inducible Systems: Based on the insect steroid hormone ecdysone and its receptor, these systems enable regulated gene expression upon addition of ecdysone or its analogs.

### Light Inducible and Heat Shock Inducible Promoters

### Galactose Inducible and Nutrient Inducible Promoters

### Oxygen-Sensitive Systems:

### Synthetic Biology Approaches:

Riboswitches: RNA elements that modulate gene expression in response to specific small molecules.
RNA-Based Systems: Utilize RNA interference (RNAi) or RNA-guided gene regulation for inducible gene expression or silencing.

Depending on the inducible system used, expression may depend on small molecules such as tetracycline, tebufenozide, 4-OHT (4-hydroxytamoxifen), doxycycline, arabinose, estradiol (or 17β-estradiol), rapamycin (or sirolimus), RU486 (mifepristone), IPTG (isopropyl β-D-1-thiogalactopyranoside), X-Gal (5-bromo-4-chloro-3-indolyl β-D-galactopyranoside), IPTG (isopropyl β-D-1-thiogalactopyranoside), AHT (anhydro-tetracycline), AAL (ara-ab-lactose), cumate, and lactose. Depending on the inducible system used, expression may depend on physical parameters such as temperature (heat), light level, and the presence of oxygen or galactose in the medium.

Table 3 provides a list of inducible elements that may be adapted and incorporated into the technology described herein.

TABLE 3
Inducible elements to control expression of tunable AAV vector modules

| Inducer Element | Inducer Molecule |
|---|---|
| CreERT2 | 4-OHT, Tamoxifen |
| EcR | Teb |
| Cumate operator | Cumate |
| TRE3G, Tet-ON | Tetracycline, doxycycline |
| QF2 | Quinic acid |
| FKBP12-ZFHD1-FRAP-p65 | Rapamycin |
| FKBP12-Gal4DBD, Cyclophilin-VP16AD, and | FKCsA |
| UAS cassettes | |
| PYL1/ABI1 (ABA activator cassette) | ABA |
| Cry2/CIB1, other photoactivatable (PA) switches | Blue light |
| Riboswitch | Aptazymes: tetracycline RNA aptamer, |
| | engineered ribozymes |
| Geneswitch | Mifepristone |

### 6. GENES TO INCLUDE THAT INHIBIT CELL DEATH INDUCED BY EXPRESSION OF VIRAL PROTEINS

Rep protein expression (Rep52 or Rep78) can induce apoptosis of an AAV infected cell line or inhibit expression of other viral genes in the absence of adenovirus. M. Schmidt et al., J Virol. 2000 October; 74 (20): 9441-9450. To prevent this effect from decreasing production of AAV, the tunable packaging system shown in FIG. 1 comprises the human gene for (Bcl-2) B-cell lymphoma 2) protein. This acts as an anti-apoptotic regulator by inhibiting the release of cytochrome c from mitochondria, thereby preventing the activation of caspases and apoptosis.

Other genes that perform a similar function and are potential substitutes for Bcl-2 in this context are Bcl-xL (B-cell lymphoma-extra large), Mcl-1 (Myeloid cell leukemia 1), Bcl-w (Bcl-2-like protein 2), Bcl-B (Bcl-2-like protein 10), Bax (Bcl-2-associated X protein), Bak (Bcl-2 homologous antagonist/killer), Bad (Bcl-2-associated death promoter), Bim (Bcl-2-interacting mediator of cell death), Bid (BH3 interacting-domain death agonist), and the BHRFI apoptosis regulator of Epstein Barr virus.

Referring to FIG. 3, activation of CreERT protein in Module B results in expression of AAV Cap, and AAV Rep52 on Module A. It also results in expression of Bcl-2 on Module C. Thus, expression of Rep 52 which may cause apoptosis, and Bcl-2 which inhibits apoptosis, are under control of the same inducible elements on both Modules A and B. More generally, any Rep gene or AAV gene that promotes apoptosis can be placed under control of any apoptosis inhibiting protein to prevent overexpression of the AAV gene from limiting AAV packaging.

### 7. USING CELL HYBRIDS AS THE HOST PACKAGING CELL

Previous disclosures by CHO Plus provide improved cell lines for manufacture of pharmaceutical agents containing viral elements, considerably reducing the cost of commercial production. See, for example, US 2024/043810 A1, entitled Cell hybrids as host cells for high efficiency production of gene therapy vectors and viral vaccines.

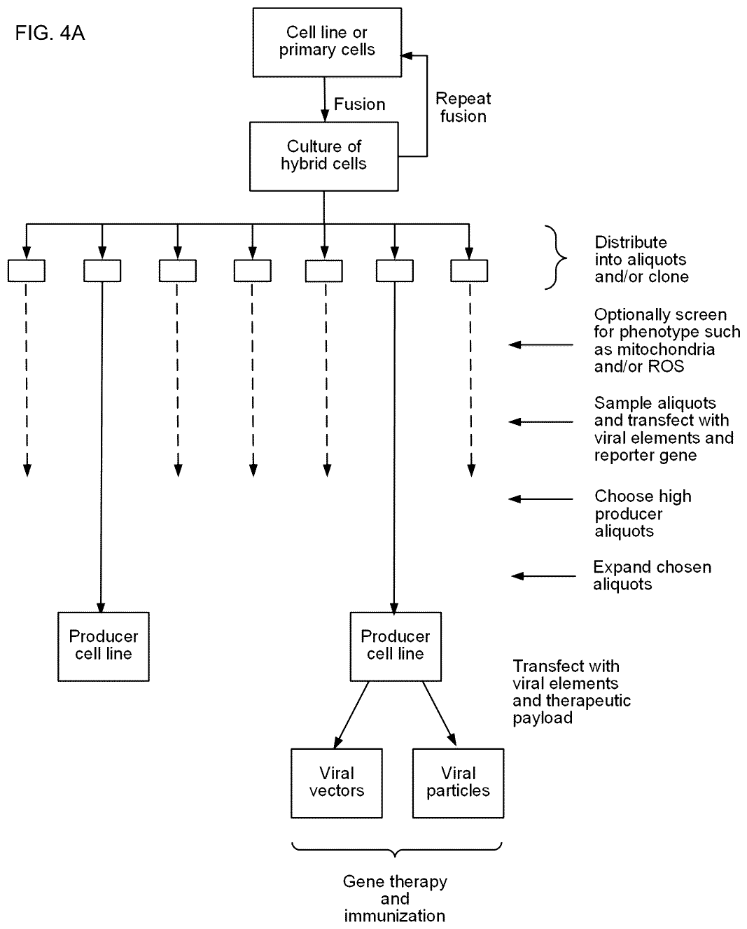

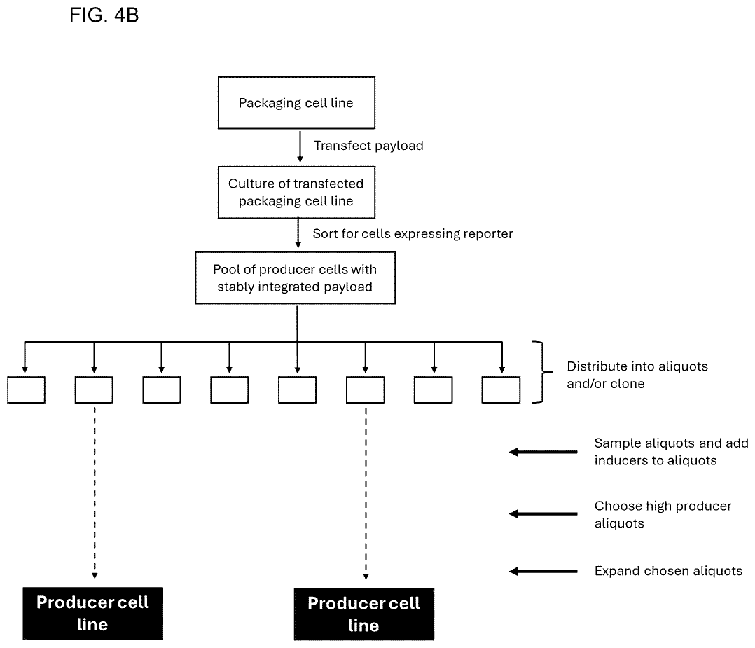

FIG. 4 is a general scheme that outlines a suitable workflow by which cell hybrids may be generated, selected, and expanded to yield AAV packaging cell lines.

Cells from a chosen source (for example, an established cell line such as HEK 293) are fused together in multiple cycles to generate a population of hybrids that are heterogeneous in their ability to synthesize viral vectors or particles. To obtain cells from the population that are high producers, the population is partitioned into a plurality of separate aliquots or clones. As part of the partitioning, the cells may be screened or separated according to particular phenotypic features that are known or suspected of being beneficial to high levels of viral capsid production or filling. A sample from each of the separate aliquots or clones are individually tested for their ability to produce high quantities or high titers of vectors or particles. Aliquots of cells that are now proven to be high producers are expanded, and used to establish one or more packaging cell lines.

The banked packaging cells can then be sourced for industrial-scale production of one or a variety of therapeutic viral vectors or particles, especially using viral components of the same species and serotype that were used for screening. The cells selected for industrial production can be transfected in the same manner used for screening, with the exception that the reporter gene is substituted with a therapeutic payload. The final transfection can be transient, or the viral elements can be integrated into the genome of the producer cell with an inducible or ubiquitous promoter-whereafter different payloads can be encapsulated into the same viral system by transient transfection.

Cell hybrids are made by obtaining a cell mixture of cells to be fused: (a plurality of cells from one cell line, or more than one cell line, or a mixture of at least one cell line and at least one primary cell population. The cell mixture is then subjected to an appropriate fusion protocol: for example, by culturing under culture conditions that promote the formation of hybrids, by conducting an electrofusion, by combining with a fusogenic virus such as Sendai virus, by placing cells into contact (for example, by gentle centrifugation), by treating with a fusogenic agent such as polyethylene glycol (PEG) or using any effective combination thereof.

Cells may be fused into hybrids using any suitable technique. For example, cells may be cultured in the presence of a fusogenic agent and/or under culture conditions that promote the formation of hybrids, or may be forced into contact, for example, by gentle centrifugation, optionally in combination with a fusogenic agent such as polyethylene glycol (PEG). Typically, a fused cell is obtained by fusing two cells together, although fusion of three or more cells is possible. It is recognized that fusion of two different cell populations will result in mixed cell products (isotopic, allotypic, or xenotypic hybrids, depending on the parental cell lines), and autotypic hybrids. Autotypic or isotopic hybrids can be separated from allotypic or xenotypic hybrids, if desired, using fluorescently labeled or surface bound antibody specific for a ligand expressed on one of the cell lines in the mixture, but not another.

### 8. DETAILED PROTOCOL FOR MAKING CELL HYBRIDS

By way of illustration (and without implying any limitation on the claimed invention and equivalents thereof), packaging cell lines for AAV vectors have been obtained according to the following protocol:

Step 1: Production of hybrids. A starting cell population of HEK 293 cells was used to make cell hybrids by using polyethylene glycol as fusogenic agent combined with gentle centrifugation to promote cell contact. Hybrids were cloned. Each clone was separated into aliquots, and sampled for transfection testing.

Step 2: Transfection. Sampled hybrid cell clones in suspension were transfected with chemical-based methods using a lipid polymer that complexes with negatively charged DNA to form lipopolyplexes via electrostatic interactions. Three plasmid vectors were used for transfection: 1) transfer vector expressing a fluorescent protein under the control of ubiquitous CMV promoter cassette flanked by AAV inverted terminal repeats: 2) helper vector cassette expressing adenovirus E4 gene for AAV DNA replication, adenovirus E2a gene and adenovirus VA RNA (virus-associated RNA) genes to enhance AAV mRNA stability and promote AAV capsid transcripts; and 3) packaging vector expressing Rep and Cap proteins specific serotype being assayed (AAV1, AAV2, and AAV5). Cells were harvested 72 hours post-transfection, lysed and assayed for AAV production.

Step 3: Determining production capability of cloned hybrids. AAV genomic copy number was measured by real-time quantitative PCR. Cell lysates were treated with DNase I to remove non-viral host genomic DNA. Real-time quantitative PCR by fluorescent detection was performed to determine viral genomic copy number. DNA primers bind to coding regions of fluorescent reporter within the transfected transfer vector in the assembled AAV and copy number was detected using fluorescence (methods used by previous figures).

Step 4: Determining AAV serotype-specific capsids. Bio-layer interferometry (BLI) is an optical biosensing technology that analyzes biomolecular interactions in real-time without the need for fluorescent labeling. Interference patterns of white light or phase shift caused by analyte sample binding to immobilized ligand on biosensor probe was used to quantify the amount of AAV virus in an unknown sample. A small biosensor that binds specifically to AAV capsid protein for multiple scrotypes (AAV1, AAV2, AAV5). For each serotype (AAV1, AAV2, and AAV5), a standard curve of a commercial AAV reference standard, with known concentration measured by other validated methods are used to back-calculate the concentration of AAV serotypes in unknown sample.

Step 5: Measuring the ratio of full to empty capsids. Ratio of full to empty capsids can be measured in unknown samples by biolayer interferometry. First, concentrations of AAV serotypes of unknown samples are measured as described above. For each serotype being measured, AAV capsids at a normalized concentration are first captured and immobilized on the biosensor. Following immobilization, AAV particles are lysed to release the packaged ssDNA and ssDNA is captured and measured using a biosensor probe that is conjugated to SSB protein wherein SSB protein binds specifically to ssDNA. For each serotype (AAV1, AAV2, and AAV5), a standard curve of a commercial AAV reference standard, with known full-to-empty capsids ratio measured by other validated methods are used to back-calculate the ratio of AAV serotypes in unknown sample.

Step 6: Measuring functional titer. For measurements of functional titer, undiluted samples of AAV1, AAV2 and AAV5 produced using the cloned hybrids were infected at a range of dilutions and added to fixed population of un-infected HEK 293 cells. Functional titers were measured in transduction units (TU) per mL. Infectivity was determined by quantifying percent fluorescent-positive cells by flow cytometry.

Step 7: Expand high producer packaging clones. The original aliquots corresponding to samples that showed high levels of capsid production and functional titer were expanded to establish producer cell lines for transduction and expression of other types of viral vectors and particles.

### 9. SELECTING HIGH PRODUCER PACKAGING CELL LINES AND PREFERRED PHENOTYPIC FEATURES

This disclosure provides a variety of means for identifying and selecting cell hybrids that have the capacity of generating high producer packaging cell lines. Cells can be transfected with a reporter gene (for example, genes that encode fluorogenic products such as green florescent protein), along with genes that encode a viral capsid for testing purposes. High producers can be selected on the basis of viral capsids produced and/or encapsulated promoter gene products.

FIG. 4B is a workflow for obtaining producer cell line for a particular AAV vector from a packaging cell line already optimized for high virus production capacity. A packaging cell line obtained according to FIG. 4A is transfected with transfer vector containing payload and a secondary fluorescent marker (such as shown in FIG. 2A). Cells are then sorted by flow cytometry for expression of secondary fluorescent reporter. Positive cells are cloned or separated into separate aliquots. Small molecule inducers that activate Modules A, B, and C are then added to separate clones or aliquots at varying concentrations and high producer cells are identified. The identified cells are expanded to establish a bank of producer cell lines. The producer cells can be used to produce AAV vectors for gene therapy or immunization without any additional transfection.

Beneficial phenotype of the packaging cell line may include a relatively high density of subcellular organelles, particularly those involved in secretion of protein or viral particles from the host cell, and the relative level or concentration of enzymes that help finish or assemble viruses. These include mitochondria, peroxisomes, endoplasmic reticulum, Golgi, and nucleoli. Such phenotypic features also include aspects of the cell cytosol or cell contents generally, such as reactive oxygen species, redox carrying molecules, and pH.

Depending on the viral system being optimized, it may be preferrable to have higher or lower levels of any of such phenotypic features, either alone or in combination. As part of the initial aliquoting or cloning step, cells can be stained with an appropriate vital dye, and separated using a cell sorter or other means into aliquots that are low (the least 10%, or the last 5% to 25%), medium (the middle 30% to 70%), or high (the greatest 10%, or the greatest 5% to 25%) in each of the features on a per-cell basis. Hybrid cells falling within any or all of these ranges can be recovered and aliquoted or cloned, then tested for virus production and effective titer. Ranges that are determined to confer an advantage can then be used as additional criteria for finding other high producer aliquots or clones for related viral serotypes or systems.

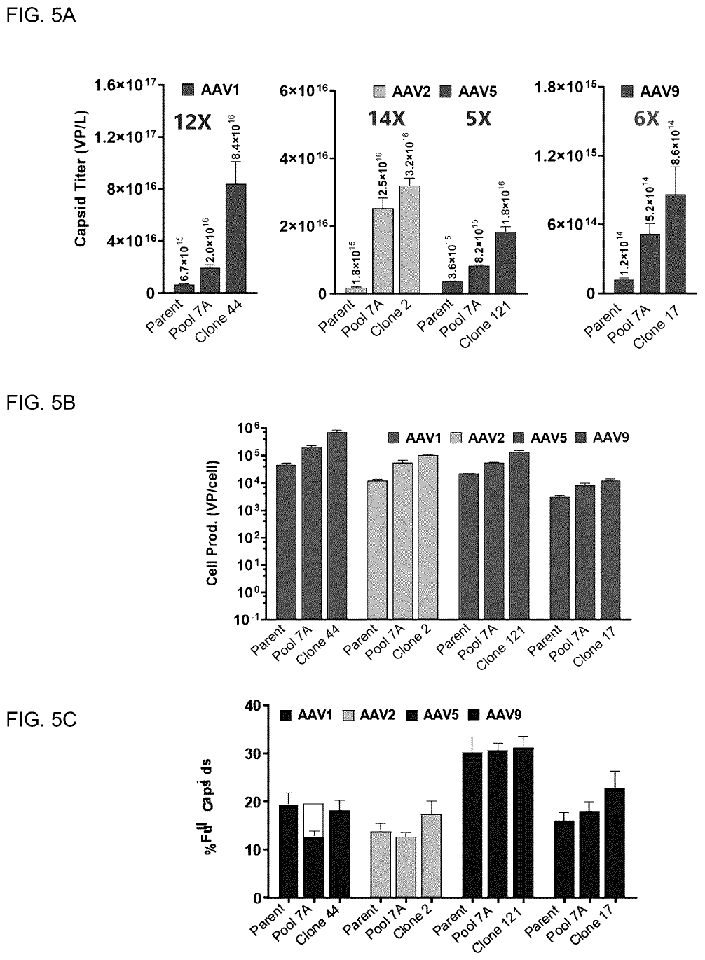

FIGS. 5A, 5B, and 5C demonstrate higher productivity from cells fused, cloned, and sampled as described. Host cells were transiently transfected with (1) transfer plasmid expressing fluorescent reporter: (2) packaging plasmid expressing Rep and Cap proteins specific for AAV1, AAV2, or AAV5; and (3) helper plasmid. Capsid concentration or titer were measured by biolayer interferometry (BLI) using a biosensor that binds to AAV1, AAV2, or AAV5 capsids. Cumulative capsids productivity (FIG. 5A), cell specific productivity (VP/cell) (FIG. 5B), and percent full capsids (FIG. 5C) of AAV1, AAV2, AAV5, AAV9 of HEK 293 parent, engineered pool (7A) and clones (44, 2, 121, and 17). Engineered clones (44, 2, 121, and 17) showed 12-fold, 14-fold, 5-fold, and 6-fold increase compared with parent host, respectively.

### 10. SELECTING FOR CELLULAR CONTENT OF MITOCHONDRIA

Virus producing cells (hybrids or standard diploid cells) can be selected for characteristic phenotypes that correlate generally with high levels of protein and/or virus product. Previous disclosures from CHO Plus demonstrate that fused cells sorted for higher amounts of mitochondria per cell and higher levels of reactive oxygen species (ROS) can be used to make producer cells that generate viral capsids that are as much as two-fold higher in the proportion of capsids that contain an intended pharmaceutical payload, such as a polynucleotide for purposes of gene therapy or vaccination.

Many viral proteins localize to the mitochondrion. Mitochondria content and function are used as basis for sorting or selection without damaging the cell using vital dyes. Such dyes can be obtained commercially, for example from the companies: Invitrogen and Sigma Aldrich. Example of vital dyes for the mitochondria include: Mito Tracker Green FM: MitoTracker Orange CMTMRos; Mito Tracker Red CMXRos: Mito Tracker Red FM: Mito Tracker Deep Red FM: BioTracker 488 Green Mitochondria dye: BioTracker 633 Red Mitochondria dye: BioTracker 405; and Blue Mitochondria.

Functional dyes to measure the membrane or redox potential of the mitochondria can also be used to sort or select for cells with enhanced mitochondria function. Mitochondria potential is generated by Complexes I. III and IV and serves as a reliable read-out to assess mitochondria function. Membrane depolarization shifts fluorescence signal from one wavelength to another. These membrane potential dyes are available from companies: Invitrogen and Sigma Aldrich: JC-1 Dye (Invitrogen T3168: Sigma CS0390): JC-9 Dye (Invitrogen D-22421); and C10 Dye (Sigma MAK160, MAK159).

Additional characteristics to sort for enhanced mitochondria includes vital dyes to measure mitochondria calcium, superoxide production, and dyes selective to the mitochondria. These include; Rhod-2 AM Reagent (Invitrogen R1245MP); and MitoSOX Red (Invitrogen M36008).

Alternatively or in addition, the user can test expression-based labeling systems that would introduce a fluorescent protein targeted to the mitochondria. They are fusion proteins comprising a portion that expresses an optical label, fused with a protein sequence that targets or is processed by the organelle to be labeled. Examples include the following. From Invitrogen: CellLight™ Mitochondria-GFP (C10600); and CellLight™ Mitochondria-RFP (C10505, C10601). From Evrogen: pTagCFP-mito (FP117); pTagYFP-mito (FP137); pTagRFP-mito (FP147); pmKate-mito (FP187); pTagGFP2-mito (FP197); pTurboRFP-mito (FP237); pTurboGFP-mito (FP517); pPhi-Yellow-mito (FP607); and pTurboFP602-mito (FP717). From Takara Bio: pAcGFP1-Mito Vector (632432); pDsRed2-Mito Vector (632421); pHcRed1-Mito Vector (632434); and pPAmCherry-Mito Vector (632591).

After staining with any of these dyes, cells may be selected (for example, by flow cytometry and sorting) that have on average a level of staining that is at least 1.2, 1.5, 2, or more than 2-fold higher than the parental cell line or lines, in terms of staining, for example, for mitochondria or an optically labeled gene product.

By way of illustration, to screen for different phenotypes of mitochondria and reactive oxygen species (ROS), hybrids were stained with CellROX® Deep Red Reagent, a fluorogenic probe for measuring cellular oxidative stress in cells: TMRM (tetramethyl rhodamine methyl ester), which measures the membrane potential of mitochondria in living cells; and Biotracker 405 Blue Mitochondria, which stains the mitochondria membrane. LIVE/DEAD Fixable NIR was used in this experiment to stain live cells.

Fused cells were cultured in complete, animal origin free (AOF), chemically defined cell culture medium: CDM4 PerMAb+6 mM L-Glutamine and detached using StemPro™ Accutase™ Cell Dissociation Reagent. Samples of cells were combined with a calculated volume of each dye to final concentrations in ˜200 mL of cell suspension containing 1×10⁸ cells in a 500 mL shake flask. Samples were incubated @; 120 rpm in shaker overnight at 37° C., with 8% CO₂.

For cell sorting, 200 mL of cell sample was centrifuged at 300×g for 5 min. The cell pellet was suspended in Accutase cell dissociation reagent, diluted, and strained into a sterile 50 mL centrifuge tubes. Cells were sorted using a Sony SH800S Cell Sorter with the following gates: Gate 1-cell ID gate; Gate 2—singlets gate; Gate 3—live cells gate; Gate 4—Biotracker 405 Blue Mitochondria (select the top 10%); Gate 5—TMRE×CellROX Deep Red (select the top 10% quadrant).

Populations of 500,000 sorted cells were expanded for 4-5 days and used for single-cell cloning in 96-well plates containing 150 μl of medium per well. Once individual wells were 80% confluent, they were expanded stepwise to 125 mL shake flasks, and used to create cell banks.

### 11. CHARACTERIZING HIGH PRODUCER CELL LINES

Cell hybrid cells that have been optimized for the production of viral vectors and particles can be characterized by one or more criteria in any combination.

Suitable criteria include cell karyotype. Chromosome patterns can be characteristic of homotypic and heterotypic cell fusions. The following characteristics may be favorable for virus production:
duplication of chromosomal segments
loss of chromosomal segments (90%, 80%, 70%, 60% or less than 50% of original segment size)
differences in heterochromatin distribution and amounts (differences of 10%, 20% or greater than 20% and/or distribution differences of heterochromatin greater than 20%)
translocation events (2 or more translocation events on the same or different chromosome segment compared to parental cell line)

Packaging or producer cells can also be characterized on the basis of cell phenotype, such as intracellular content of mitochondria, peroxisomes, reactive oxygen species (ROS), endoplasmic reticulum, Golgi apparatus, nucleoli, and so on, using the materials provided above.

### 12. DETERMINING PRODUCTION CAPACITY AND CHARACTERISTICS OF PRODUCER CELLS

A cell line or mixed cell population that has been selected for high levels of virus production may be characterized in comparison with the parental or originating cell line by any one or more of several different parameters. For example, the selected cells may have: (1) a genome that is more aneuploid than the starting cells, containing part or all of the genome of two or more parental cell lines (which may or may not be the same), (2) a higher concentration of mitochondria, peroxisomes, endoplasmic reticulum, Golgi apparatus, reactive oxygen species, or other phenotypic feature compared with any one or all of the parental cell lines (for example, between 2 to 5-fold or 4 to 8 fold, or more than 2-, 4-, or 8-fold higher), (3) a capacity to produce a level of virus per cell or per liter of culture fluid that is substantially higher than the parental cell line (for example, between 2 to 5-fold or 4 to 8 fold, or more than 2-, 4-, or 8-fold higher), (4) a capacity to produce a particular amount of virus per cell (for example, more than 50, 65, 75, 100, 150, 200, 300, 500, 2000, 5000, or 20,000 capsids per cell; (5) a capacity to produce a certain amount of virus per volume of culture fluid (for example, at least 5, 8, 12, 20, or 30 grams, or between 8 and 20 or between 10 and 50 grams of virus per liter of culture fluid: or (6) a capacity to produce viral vectors or particles that have a higher proportion of payload-carrying capsids (50% higher, or 2 or 3-fold).

For the purpose of making such comparisons, the producer cell line can be compared with a standardized population of the original cell line, either kept on hand, as part of the same system, or obtained from a reference source. For example, CHO derived producer cells may be compared with CRL-12023 cells from the American Type Culture Collection (ATCC®). This disclosure includes systems for high-level production of virus-based pharmaceuticals, comprising both a starting cell line, and a producer cell line derived therefrom that has a relatively high density of endoplasmic reticulum and/or Golgi apparatus per cell, as determined, for example, using one or more of the vital dyes listed above.

### 13. GENETICALLY ALTERING PRODUCER CELLS TO SYNTHESIZE AND PRODUCE VIRAL ELEMENTS

Adeno-associated viruses (AAV) can be produced by transient transfection of one or more combinations of the following vectors into a cell line: helper, packaging, envelope, and/or transfer vectors, Gene cassette for helper, packaging, envelope and transfer vectors differ depending on the type of virus produced. Helper vector can express E2A and E4 genes as well as the VA RNA for adenovirus and AAV. In another illustration, packaging vector expresses Rep and Cap genes for adenovirus and AAV while a different packaging vector expresses Gag, Pol, Rev and its response elements for lentivirus production

Alternatively. Rep or envelope genes can be expressed in an inducible manner wherein Rep gene cassette is split in two segments (5′ and 3′ segment) and these two segments are joined by stop cassette, containing transcription termination and polyadenylation sequences flanked by two homologous recombination sites, located in cis. In another preferred illustration, E2, E4 and VA cassette is placed under the control of an inducible promoter in the reverse orientation with respect to the promoter. Activation of EEV is achieved through delivery of Cre and doxycycline. In a preferred illustration, loxP and lox511 is used for recombination. Other heterologous recombination sites can be used: lox2272, FRT, and mFRT71. In the case that any combination of FRT and mFRT71 recombination sites are used. Flippase must also be delivered to the cell. Envelope vector is only required for production of lentiviruses not adenovirus or AAV. Transfer vectors backbone containing gene of interest is unique to virus type. Lentivirus transfer vectors backbone consists of: 5′ and 3′ LTR. Psi packaging signal, and Woodchuck Hepatitis Virus Posttranscriptional Regulatory Element (WPRE). Adenovirus and AAV transfer vector backbone contain inverted terminal repeats flanking promoter, gene of interest, and WPRE.

AAV can be produced by transfection of helper, packaging, transfer vectors into a cell line which does not contain integrated copies of any of the viral vectors. In another illustration, transfer vector (expressing gene of interest) is transfected in a cell line containing stable integration of helper and packaging elements. In another illustration, the cell line may have stable integration of helper, packaging and transfer vectors.

Transfection can be done using liposome-based reagents (for example. Lipofectamine™ 3000. Expifectamine 293. FuGENE™ HD. X-Fect nanoparticles polymer. Trans-IT Pro reagents. Trans-IT VirusGen, polyethylenimine), calcium phosphate, electroporation, or infection with an adenovirus, retrovirus or lentivirus-based vector.

Following transfection, the cells are tested for production for packaging of the intended virus: for example, by enzyme-linked immunosorbent assay (ELISA), quantitative real-time PCR (qPCR), or biolayer interferometry (BLI). Cells or clones having increased production of the desired virus are selected. The objective can be an increase in virus production that is 1.5, 2, 4, 8, 12, 16, 20, or 100-fold higher than the parental cell line; and/or production at a level of greater than 10¹² viral genome/ml or capsids/ml for AAV; and/or greater than 10⁸ infectious units (IFU) per ml of culture fluid under typical manufacturing conditions. The virus of interest can also be tested for other desired characteristics, such as full to empty capsid ratio and functional titer.

In principle, the transfection can be done either before, during, or after one or more cycles of fusion of the packaging cell line and selection for other features. For example, the fusion and selection can be done before transfection with the packaging, helper and transfer vector containing gene of interest, thereby establishing a parental cell line suitable for high-level of virus production of the user's choice. Alternatively, the transfection can be done into the originating parental cell line containing gene(s) of interest and used to track production levels during subsequent fusion and sorting steps, or to provide another basis for such sorting. Alternatively, the transfection can be done as an intermediate step, wherein the cells have already been subject to one or more cycles of fusion and selection for some other feature such as mitochondria and ROS. The resulting hybrid is transfected to express virus of interest, and then subjected to further cycles of fusion and selection for expression of the virus of interest and/or other features referred to earlier in this disclosure.

Another option is to develop a cell line using a reporter gene as a proxy for the virus payload that ultimately will be manufactured: for example, secreted alkaline phosphatase, secreted luciferase, fluorescent virus payloads such as red fluorescent virus payload or green fluorescent virus payload. Again, the transfection can be done before, during, or after multiple cycles of fusion and selection, optionally using the level of expression of the marker as the selection criteria in one or more of the cycles. This creates a parental cell line that is optimized for expression of the marker virus payload, with the expectation that the beneficial characteristics of the cell line will be retained after further genetic alteration to produce a biological product of commercial interest.

Ultimately, once a cell line has been developed having a desired level of expression of the marker virus payload, the marker is then replaced with the virus payload of interest. Transfection can again be done randomly into the genome, using the techniques listed above, and expression of the reporter gene is curtailed. Alternatively, the gene for the reporter gene can be substituted with a gene that encodes the virus payload of interest using a targeted integration technique. Such techniques comprise, for example. CRISPR/Cas virus payloads. CRISPR/Cas associated transposase (CASTs), recombinase cassette exchange (RMCE), a zinc-finger recombinase (ZFR), serine integrases, or a transcription activator-like effector nuclease (TALEN). That way, the gene of interest is inserted into the genome of the cells from the producer cell line or the mixture at a location that is pre-selected as permitting or supporting a high level of transcription, compared with other locations in the genome.

### 14. METHODS FOR QUANTIFYING LEVELS OF VIRAL PRODUCTION FROM CELL HYBRIDS

Real-time quantitative PCR measures viral transcription, concentration of viral genome (vg/ml). Each viral particle typically contains one viral genome. Viruses are treated with DNase I to remove any of the host genomic DNA. Primers binding to targeted regions in the transfer vector are used and amplicon is detected by either probe-based method or SYBR Green, which binds to the amplicon.

Indirect ELISA and biolayer interferometry (BLI) are used to measure total capsid AAV particles. These measurements utilize an antibody against an abundant capsid protein present in AAV serotype. Samples are captured by capsid antibody and detected using biotinylated capsid antibody and Streptavidin conjugated to HRP for chemiluminescent detection.

For lentivirus, infectious particles can be measured by indirect or sandwich ELISA using antibody to p24. Anti-p24 is used to capture samples and detected using biotinylated anti-p24 along with Streptavidin conjugated to HRP for chemiluminescent detection.

Functional titer or infectious titer of viruses is the concentration of viral particles that can transduce cells. Functional titer can be measured by cell transduction using a fluorescent or chemiluminescent protein as a reporter. Cell lines are infected or transduced with packaged viruses at specific multiplicity of infections (MOI). % of cells expressing reporter gene are quantified and correlated with the #of virus particles used to transduce cells.

### 15. PHARMACEUTICAL PAYLOADS AND THERAPEUTIC APPLICATIONS

Viral vectors and particles produced according to this disclosure can be used for delivering a variety of pharmaceutical payloads to human subjects in need thereof. Vectors made from adeno-associated virus (AAV), adenovirus (AdV), lentivirus (LV), retrovirus (RV), and herpes simplex virus (HSV) may be suitable, depending on context. Treatment is done by administering to a subject an amount of the vector or particle that is effective in achieving one or more clinical aims. Some examples are provided in Table 1:

TABLE 4
Vector types and therapeutic targets

| Virus | Benefits | Example | Exemplary Strain |
|---|---|---|---|
| adeno- | low immunogenicity of vector; | luxturna (voretigene | AAV2, AAV9 |
| associated | long-term gene expression; | neparvovec) for inherited | |
| virus (AAV) | wide tissue tropism | retinal disease | |
| lentivirus and | stable integration into the | strimvelis for ADA-SCID | HIV-1-based lentivirus |
| retrovirus | host genome; | (adenosine deaminase | such as VSV-G- |
| | suitable for dividing and non- | deficiency severe | pseudotyped HIV-1 |
| | dividing cells; | combined | |
| | large cargo capacity | immunodeficiency) | |
| adenovirus | efficient gene transfer; | cancer, infectious | Adenovirus serotype 5 |
| | large cargo capacity; | diseases, and genetic | (Ad5), Adenovirus |
| | broad tropism; | disorders | serotype 26 (Ad26) |
| | proven safety | | |
| Herpes | large DNA cargo capacity; | glioblastoma multiforme | HSV-1, HSV-2 |
| simplex virus | neuronal transduction | and neurological | |
| (HSV) | capabilities | disorders | |
| vaccinia virus | large genome capacity; | cancer immunotherapy | Modified vaccinia virus |
| | broad cell tropism; | | Ankara (MVA), Wyeth |
| | strong immunogenicity | | strain |

The technology of this disclosure is advantageous for delivering a nucleic acid, a protein, or mixture thereof for purposes of inducing a specific immunological response. The packaged nucleic acid encodes one or more epitopes from the intended immune target, and optionally one or more additional proteins that may act as an adjuvant or stimulant to enhance immunogenicity. The target may be an infectious agent, such as a pathogenic virus, bacteria, or protozoan. Alternatively, the target may be a cancer cell, in which case the encoded epitopes are epitopes expressed by the cancer cell that are specific to the cancer or to the tissue type. For example, the technology of this disclosure can be used to prepare a composition to induce a response to the SARS-COV-2 virus, for the purpose of prevention or treatment of COVID-19. Representative immunogenic epitopes may be taken from any one or more of the four SARS-COV-2 structural proteins: namely, membrane glycoprotein (M), envelope protein (E), nucleocapsid protein (N), and the spike protein(S). Most current vaccines against SARS-COV-2 typically include or encode the whole spike protein. Ways to optimize the spike protein were recently discussed by F. Heinz & K. Stiasny, NPJ Vaccines (2021) 6:104.

The technology of this disclosure can also be used for the purpose of gene therapy: for example, delivery of a nucleic acid encoding a gene product that is missing or defective in the subject being treated, or targeted to pathogenic cells in the subject, particularly cancer cells. Therapeutic purposes include but are not limited to expression of a therapeutic protein encoded in the nucleic acid (such as a cytokine or anti-cancer agent), expression of an essential protein that the subject is unable to produce themselves, or delivery of a gene editing system such as CRISPR/Cas9 or a guide RNA. Other possible therapeutic payloads may include DNA antisense oligonucleotides, DNA aptamers: messenger RNAs, micro RNAs, short interfering RNAs, ribozymes, RNA decoys and circular RNAs that specifically increase or decrease expression of a particular endogenous gene in the subject or an infectious agent. K. Sridharan et al., Br J Clin Pharmacol. 2016 September: 82 (3): 659-672.

### 16. MEDICAMENTS AND COMMERCIAL PRODUCTS

Preparation and formulation of pharmaceutical agents for use according to this disclosure can incorporate standard technology, as described, for example, in the most recent edition of Remington: The Science and Practice of Pharmacy. The formulation will typically be optimized for administration systemically, either intramuscularly or subcutaneously, or for administration orally or nasally (for example, to stimulate the mucosal immune system).

Preparations of viral vectors and particles may be provided as one or more unit doses (either combined or separate), each containing an amount of the pharmaceutical payload that is effective in the treatment of a chosen disease, infection, or clinical condition. The commercial product may contain a device such as a syringe for administration of the agent or composition in or around the target tissue of a subject in need thereof. The product may also contain or be accompanied by an informational package insert describing the use and attendant benefits of the vector or particle in treating the condition for which it is indicated and approved.

### 17. INCORPORATION BY REFERENCE

For all purposes in the United States of America, each and every publication and patent document referred to in this disclosure is incorporated herein by reference in its entirety for all purposes to the same extent as if each such publication or document was specifically and individually indicated to be incorporated herein by reference.

### 18. PRACTICE OF THE CLAIMED INVENTION

The technology provided in this disclosure and its use are described within a hypothetical understanding of general principles of virus and pharmaceutical manufacture. These discussions are provided for the edification and interest of the reader, and are not intended to limit the practice of the claimed invention. All of the products and methods claimed in this application may be used for any suitable purpose without restriction, unless otherwise indicated or required.

While this disclosure has been described with reference to the specific embodiments, changes can be made and equivalents can be substituted to adapt this disclosure to a particular context or intended use as a matter of routine experimentation, thereby achieving benefits of this disclosure without departing from the scope of what is claimed.

The invention claimed is:

1. A tunable adeno-associated virus (AAV) vector packaging cell line, comprising recombinantly inserted transgenes in at least three modules:
a first module, which comprises at least one AAV cap gene under control of a first inducible regulatory element;
a second module, which comprises one or a plurality of AAV helper genes under control of a second inducible regulatory element; and
a third module, which comprises at least one AAV rep gene under control of a third inducible regulatory element.

2. The tunable packaging cell line of claim 1, further comprising a payload module that contains an expressible gene encoding a vector payload between two AAV inverted terminal repeat sequences (ITRs).

3. The tunable packaging cell line of claim 1, further comprising a gene that encodes an apoptosis inhibitor protein under control of an inducible regulatory element.

4. The tunable packaging cell line of claim 3, wherein the apoptosis inhibitor protein is Bcl-2.

5. The tunable packaging cell line of claim 1, wherein at least one of said regulatory elements is an ecdysone receptor fusion protein.

6. The tunable packaging cell line of claim 1, wherein the first inducible regulatory element controls a plurality of AAV cap genes and at least one AAV rep gene.

7. The tunable packaging cell line of claim 6, wherein said cap genes and rep gene are also under control of the second inducible regulatory element.

8. The tunable packaging cell line of claim 1, wherein at least one of said regulatory elements is a recombinase enzyme with a ligand binding domain.

9. The tunable packaging cell line of claim 1, wherein the second regulatory element activates expression of the AAV helper genes by inversion.

10. The tunable packaging cell line of claim 6, wherein the second regulatory element activates expression of said cap genes and rep gene by inversion.

11. The tunable packaging cell line of claim 3, wherein the gene that encodes the apoptosis inhibitor protein is under control of both the first and second inducible regulatory element.

12. The tunable packaging cell line of claim 1, wherein at least one of said regulatory elements is a reverse tetracycline-controlled transactivator (rtTA).

13. The tunable packaging cell line of claim 1, wherein the first regulatory element is an ecdysone receptor fused to a DNA binding domain, the second regulatory element is a Cre recombinase enzyme with a ligand binding domain, and the third regulatory element is a reverse tetracycline-controlled transactivator (rtTA).

14. The tunable packaging cell line of claim 2, wherein the vector payload is a marker protein such as green fluorescent protein (GFP).

15. The tunable packaging cell line of claim 2, wherein the vector payload is an immunogenic peptide or a nucleic acid encoding said peptide for eliciting a specific immune response in a subject in need thereof.

16. The tunable packaging cell line of claim 2, wherein the vector payload is a nucleic acid encoding a gene product that is deficient in a subject in need of gene therapy therefor.

17. The tunable packaging cell line of claim 1, which is the progeny of a fusion of two or more parental cell lines.

18. The tunable packaging cell line of claim 1, which is the progeny of a fusion of two HEK-293 cells.

19. The tunable packaging cell line of claim 17, wherein the progeny have been selected for amount of mitochondria content or reactive oxygen species per cell.

20. A method of optimizing production of an AAV vector, comprising:
(a) transfecting the tunable packaging cell line of claim 1 with a payload vector comprising a gene that encodes a vector payload between two AAV inverted terminal repeat sequences (ITRs);
(b) culturing cells transfected in step (a) or progeny thereof in the presence of different amounts and/or ratios of inducer compounds for each of said three inducible regulatory elements, thereby producing said AAV vector; and
(c) adjusting the amounts and/or the ratios of the inducer compounds to obtain a desired titer of AAV serotype-specific capsids (viral particles/mL); ratio of full to empty capsids, and/or functional titer (transduction units/mL).

21. A method of producing an AAV vector, comprising culturing the tunable packaging cell line of claim 1 in a medium containing a predetermined amount of each inducer compound for each of said three inducible regulatory elements; and harvesting the AAV vector from the medium.
